(19) United States
(12) Patent Application Publication
(10) Pub. No.: US 2014/0213648 A1
(43) Pub. Date: Jul. 31, 2014

(54) METHODS OF INCREASING EPA BLOOD LEVELS

(71) Applicant: Amarin Pharmaceuticals Ireland Limited, Dublin (IE)

(72) Inventors: Mehar Manku, Birmingham (GB); Ian Osterloh, Kent (GB); Pierre Wicker, Mystic, CT (US); Rene Braeckman, Richboro, PA (US); Paresh Soni, Mystic, CT (US)

(73) Assignee: Amarin Pharmaceuticals Ireland Limited, Dublin (IE)

(21) Appl. No.: 14/145,030

(22) Filed: Dec. 31, 2013

Related U.S. Application Data
(60) Provisional application No. 61/747,689, filed on Dec. 31, 2012.

(51) Int. Cl. A61K 31/202

(52) U.S. Cl. CPC A61K 31/202
USPC 514/560

(57) ABSTRACT

In various embodiments, the present invention provides methods of treating and/or preventing cardiovascular-related disease and, in particular, a method of blood lipid therapy comprising administering to a subject in need thereof a pharmaceutical composition comprising eicosapentaenoic acid or a derivative thereof.

### PRIORITY CLAIM

This application claims priority to U.S. Provisional Patent Application No. 61/747,689, filed on Dec. 31, 2012, the entire contents of which are incorporated herein by reference and relied upon.

### BACKGROUND

Cardiovascular disease is one of the leading causes of death in the United States and most European countries. It is estimated that over 70 million people in the United States alone suffer from a cardiovascular disease or disorder including but not limited to high blood pressure, coronary heart disease, dyslipidemia, congestive heart failure and stroke. A need exists for improved treatments for cardiovascular diseases and disorders.

### SUMMARY

In various embodiments, the present invention provides methods of treating and/or preventing cardiovascular-related diseases and, in particular, a method of blood lipid therapy comprising administering to a subject in need thereof a pharmaceutical composition comprising eicosapentaenoic acid or a derivative thereof. In one embodiment, the composition contains not more than 10%, by weight, docosahexaenoic acid or derivative thereof, substantially no docosahexaenoic acid or derivative thereof, or no docosahexaenoic acid or derivative thereof. In another embodiment, eicosapentaenoic acid ethyl ester comprises at least 90%, by weight, of all fatty acids present in the composition; the composition contains not more than 10%, by weight, of total fatty acids other than eicosapentaenoic acid ethyl ester; and/or the composition contains about 0.1% to about 0.6% of at least one fatty acid other than eicosapentaenoic acid ethyl ester and docosahexaenoic acid (or derivative thereof).

In one embodiment, a pharmaceutical composition useful in accordance with the invention comprises, consists of or consists essentially of at least 95% by weight ethyl eicosapentaenoate (EPA-E), about 0.2% to about 0.5% by weight ethyl octadecatetraenoate (ODTA-E), about 0.05% to about 0.25% by weight ethyl nonadecapentaenoate (NDPA-E), about 0.2% to about 0.45% by weight ethyl arachidonate (AA-E), about 0.3% to about 0.5% by weight ethyl eicosatetraenoate (ETA-E), and about 0.05% to about 0.32% ethyl heneicosapentaenoate (HPA-E). In another embodiment, the composition is present in a capsule shell. In another embodiment, the composition contains substantially no or no amount of docosahexaenoic acid (DHA) or derivative thereof such as ethyl-DHA (DHA-E).

In another embodiment, the invention provides a method of treating moderate to severe hypertriglyceridemia comprising administering a composition as described herein to a subject in need thereof one to about four times per day.

These and other embodiments of the present invention will be disclosed in further detail herein below.

### BRIEF DESCRIPTION OF THE DRAWINGS

FIG. 1 depicts the mean placebo-adjusted changes from baseline in plasma and red blood cell concentrations of EPA after 12 weeks of treatment with 2 g/day or 4 g/day of ultra-pure EPA.

FIG. 2A depicts placebo-adjusted least square mean changed from baseline in plasma and red blood cell concentrations and ratios of selected fatty acids and fatty acid groups for 4 g/day doses of ultra-pure EPA. FIG. 2B depicts placebo-adjusted least square mean changed from baseline in plasma and red blood cell concentrations and ratios of selected fatty acids and fatty acid groups for 2 g/day doses of ultra-pure EPA.

FIG. 3 depicts the dose dependence of EPA concentrations in plasma and red blood cells.

FIG. 4A depicts the relationship between plasma triglyceride-lowering and EPA concentrations in plasma (a pharmacokinetic/pharmacodynamic relationship).

FIG. 4B depicts the relationship between plasma triglyceride-lowering and EPA concentrations in red blood cells (a pharmacokinetic/pharmacodynamic relationship).

### DETAILED DESCRIPTION

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” Also, the disclosure of ranges is intended as a continuous range including every value between the minimum and maximum values recited as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present invention.

In one embodiment, the invention provides a method for treatment and/or prevention of a cardiovascular-related disease. The term “cardiovascular-related disease” herein refers to any disease or disorder of the heart or blood vessels (i.e. arteries and veins) or any symptom thereof. Non-limiting examples of cardiovascular-related disease and disorders include hypertriglyceridemia, hypercholesterolemia, mixed dyslipidemia, coronary heart disease, vascular disease, stroke, atherosclerosis, arrhythmia, hypertension, myocardial infarction, and other cardiovascular events.

The term “treatment” in relation a given disease or disorder, includes, but is not limited to, inhibiting the disease or disorder, for example, arresting the development of the disease or disorder; relieving the disease or disorder, for example, causing regression of the disease or disorder; or relieving a condition caused by or resulting from the disease or disorder, for example, relieving, preventing or treating symptoms of the disease or disorder. The term “prevention” in relation to a given disease or disorder means: preventing the onset of disease development if none had occurred, preventing the disease or disorder from occurring in a subject that may be predisposed to the disorder or disease but has not yet been diagnosed as having the disorder or disease, and/or preventing further disease/disorder development if already present.

In one embodiment, the present invention provides a method of blood lipid therapy comprising administering to a subject or subject group in need thereof a pharmaceutical composition as described herein. In another embodiment, the subject or subject group has hypertriglyceridemia, hypercholesterolemia, mixed dyslipidemia and/or very high triglycerides.

In another embodiment, the subject or subject group being treated has a baseline triglyceride level (or median baseline triglyceride level in the case of a subject group), fed or fasting, of at least about 300 mg/dl, at least about 400 mg/dl, at least about 500 mg/dl, at least about 600 mg/dl, at least about 700 mg/dl, at least about 800 mg/dl, at least about 900 mg/dl, at least about 1000 mg/dl, at least about 1100 mg/dl, at least about 1200 mg/dl, at least about 1300 mg/dl, at least about 1400 mg/dl, or at least about 1500 mg/dl, for example about 400 mg/dl to about 2500 mg/dl, about 450 mg/dl to about 2000 mg/dl or about 500 mg/dl to about 1500 mg/dl.

In one embodiment, the subject or subject group being treated in accordance with methods of the invention has previously been treated with Lovaza® and has experienced an increase in, or no decrease in, LDL-C levels and/or non-HDL-C levels. In one such embodiment, Lovaza® therapy is discontinued and replaced by a method of the present invention.

In another embodiment, the subject or subject group being treated in accordance with methods of the invention exhibits a fasting baseline absolute plasma level of free EPA (or mean thereof in the case of a subject group) not greater than about 0.70 nmol/ml, not greater than about 0.65 nmol/ml, not greater than about 0.60 nmol/ml, not greater than about 0.55 nmol/ml, not greater than about 0.50 nmol/ml, not greater than about 0.45 nmol/ml, or not greater than about 0.40 nmol/ml. In another embodiment, the subject or subject group being treated in accordance with methods of the invention exhibits a baseline fasting plasma level (or mean thereof) of free EPA, expressed as a percentage of total free fatty acid, of not more than about 3%, not more than about 2.5%, not more than about 2%, not more than about 1.5%, not more than about 1%, not more than about 0.75%, not more than about 0.5%, not more than about 0.25%, not more than about 0.2% or not more than about 0.15%. In one such embodiment, free plasma EPA and/or total fatty acid levels are determined prior to initiating therapy.

In another embodiment, the subject or subject group being treated in accordance with methods of the invention exhibits a fasting baseline absolute plasma level of total fatty acid (or mean thereof) not greater than about 250 nmol/ml, not greater than about 200 nmol/ml, not greater than about 150 nmol/ml, not greater than about 100 nmol/ml, or not greater than about 50 nmol/ml.

In another embodiment, the subject or subject group being treated in accordance with methods of the invention exhibits a fasting baseline plasma, serum or red blood cell membrane EPA level not greater than about 70 μg/ml, not greater than about 60 μg/ml, not greater than about 50 μg/ml, not greater than about 40 μg/ml, not greater than about 30 μg/ml, or not greater than about 25 μg/ml.

In another embodiment, methods of the present invention comprise a step of measuring the subject's (or subject group's mean) baseline lipid profile prior to initiating therapy. In another embodiment, methods of the invention comprise the step of identifying a subject or subject group having one or more of the following: baseline non-HDL-C value of about 200 mg/dl to about 400 mg/dl, for example at least about 210 mg/dl, at least about 220 mg/dl, at least about 230 mg/dl, at least about 240 mg/dl, at least about 250 mg/dl, at least about 260 mg/dl, at least about 270 mg/dl, at least about 280 mg/dl, at least about 290 mg/dl, or at least about 300 mg/dl; baseline total cholesterol value of about 250 mg/dl to about 400 mg/dl, for example at least about 260 mg/dl, at least about 270 mg/dl, at least about 280 mg/dl or at least about 290 mg/dl; baseline vLDL-C value of about 140 mg/dl to about 200 mg/dl, for example at least about 150 mg/dl, at least about 160 mg/dl, at least about 170 mg/dl, at least about 180 mg/dl or at least about 190 mg/dl; baseline HDL-C value of about 10 to about 60 mg/dl, for example not more than about 40 mg/dl, not more than about 35 mg/dl, not more than about 30 mg/dl, not more than about 25 mg/dl, not more than about 20 mg/dl, or not more than about 15 mg/dl; and/or baseline LDL-C value of about 50 to about 300 mg/dl, for example not less than about 100 mg/dl, not less than about 90 mg/dl, not less than about 80 mg/dl, not less than about 70 mg/dl, not less than about 60 mg/dl or not less than about 50 mg/dl.

In a related embodiment, upon treatment in accordance with the present invention, for example over a period of about 1 to about 200 weeks, about 1 to about 100 weeks, about 1 to about 80 weeks, about 1 to about 50 weeks, about 1 to about 40 weeks, about 1 to about 20 weeks, about 1 to about 15 weeks, about 1 to about 12 weeks, about 1 to about 10 weeks, about 1 to about 5 weeks, about 1 to about 2 weeks or about 1 week, the subject or subject group exhibits one or more of the following outcomes:

(a) reduced triglyceride levels compared to baseline;

(b) reduced Apo B levels compared to baseline;

(c) increased HDL-C levels compared to baseline;

(d) no increase in LDL-C levels compared to baseline;

(e) a reduction in LDL-C levels compared to baseline;

(f) a reduction in non-HDL-C levels compared to baseline;

(g) a reduction in vLDL levels compared to baseline;

(h) an increase in apo A-I levels compared to baseline;

(i) an increase in apo A-I/apo B ratio compared to baseline;

(j) a reduction in lipoprotein A levels compared to baseline;

(k) a reduction in LDL particle number compared to baseline;

(l) an increase in LDL size compared to baseline;

(m) a reduction in remnant-like particle cholesterol compared to baseline;

(n) a reduction in oxidized LDL compared to baseline;

(o) no change or a reduction in fasting plasma glucose (FPG) compared to baseline;

(p) a reduction in hemoglobin A_{1c} (HbA_{1c}) compared to baseline;

(q) a reduction in homeostasis model insulin resistance compared to baseline;

(r) a reduction in lipoprotein associated phospholipase A2 compared to baseline;

(s) a reduction in intracellular adhesion molecule-1 compared to baseline;

(t) a reduction in interleukin-6 compared to baseline;

(u) a reduction in plasminogen activator inhibitor-1 compared to baseline;

(v) a reduction in high sensitivity C-reactive protein (hsCRP) compared to baseline;

(w) an increase in serum or plasma EPA compared to baseline;

(x) an increase in red blood cell (RBC) membrane EPA compared to baseline;

(y) a reduction or increase in one or more of serum phospholipid and/or red blood cell content of docosahexaenoic acid (DHA), docosapentaenoic acid (DPA), arachidonic acid (AA), palmitic acid (PA), staeridonic acid (SA) or oleic acid (OA) compared to baseline;

(z) a reduction in the ratio of arachidonic acid to EPA (“AA/EPA”) in serum, plasma, and/or RBCs compared to baseline or placebo control;

(aa) an increase in the ratio of omega-3 fatty acids to omega-6 fatty acids in serum, plasma, and/or RBCs compared to baseline or placebo control;

(bb) a decrease in the ratio of omega-6 fatty acids to total fatty acids in serum, plasma, and/or RBCs compared to baseline or placebo control; and/or

(cc) an increase in the ratio of omega-3 fatty acids to total fatty acids in serum, plasma, and/or RBCs compared to baseline or placebo control.

In one embodiment, upon administering a composition of the invention to a subject, the subject exhibits a decrease in triglyceride levels, an increase in the concentrations of EPA and DPA (n-3) in red blood cells, and an increase of the ratio of EPA:arachidonic acid in red blood cells. In a related embodiment the subject exhibits substantially no or no increase in RBC DHA.

In one embodiment, methods of the present invention comprise measuring baseline levels of one or more markers set forth in (a)-(cc) above prior to dosing the subject or subject group. In another embodiment, the methods comprise administering a composition as disclosed herein to the subject after baseline levels of one or more markers set forth in (a)-(cc) are determined, and subsequently taking an additional measurement of said one or more markers.

In another embodiment, upon treatment with a composition of the present invention, for example over a period of about 1 to about 200 weeks, about 1 to about 100 weeks, about 1 to about 80 weeks, about 1 to about 50 weeks, about 1 to about 40 weeks, about 1 to about 20 weeks, about 1 to about 15 weeks, about 1 to about 12 weeks, about 1 to about 10 weeks, about 1 to about 5 weeks, about 1 to about 2 weeks or about 1 week, the subject or subject group exhibits any 2 or more of, any 3 or more of, any 4 or more of, any 5 or more of, any 6 or more of, any 7 or more of, any 8 or more of, any 9 or more of, any 10 or more of, any 11 or more of, any 12 or more of, any 13 or more of, any 14 or more of, any 15 or more of, any 16 or more of, any 17 or more of, any 18 or more of, any 19 or more of, any 20 or more of, any 21 or more of, any 22 or more of, any 23 or more, any 24 or more, any 25 or more, any 26 or more, any 27 or more, any 28 or more, or all 29 of outcomes (a)-(cc) described immediately above.

In another embodiment, upon treatment with a composition of the present invention, the subject or subject group exhibits one or more of the following outcomes:

(a) a reduction in triglyceride level of at least about 5%, at least about 10%, at least about 15%, at least about 20%, at least about 25%, at least about 30%, at least about 35%, at least about 40%, at least about 45%, at least about 50%, at least about 55% or at least about 75% (actual % change or median % change) as compared to baseline;

(b) a less than 30% increase, less than 20% increase, less than 10% increase, less than 5% increase or no increase in non-HDL-C levels or a reduction in non-HDL-C levels of at least about 1%, at least about 3%, at least about 5%, at least about 10%, at least about 15%, at least about 20%, at least about 25%, at least about 30%, at least about 35%, at least about 40%, at least about 45%, at least about 50%, at least about 55% or at least about 75% (actual % change or median % change) as compared to baseline;

(c) substantially no change in HDL-C levels, no change in HDL-C levels, or an increase in HDL-C levels of at least about 5%, at least about 10%, at least about 15%, at least about 20%, at least about 25%, at least about 30%, at least about 35%, at least about 40%, at least about 45%, at least about 50%, at least about 55% or at least about 75% (actual % change or median % change) as compared to baseline;

(d) a less than 60% increase, a less than 50% increase, a less than 40% increase, a less than 30% increase, less than 20% increase, less than 10% increase, less than 5% increase or no increase in LDL-C levels or a reduction in LDL-C levels of at least about 5%, at least about 10%, at least about 15%, at least about 20%, at least about 25%, at least about 30%, at least about 35%, at least about 40%, at least about 45%, at least about 50%, at least about 55%, at least about 55% or at least about 75% (actual % change or median % change) as compared to baseline;

(e) a decrease in Apo B levels of at least about 5%, at least about 10%, at least about 15%, at least about 20%, at least about 25%, at least about 30%, at least about 35%, at least about 40%, at least about 45%, at least about 50%, at least about 55% or at least about 75% (actual % change or median % change) as compared to baseline;

(f) a reduction in vLDL levels of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(g) an increase in apo A-I levels of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(h) an increase in apo A-I/apo B ratio of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(i) a reduction in lipoprotein (a) levels of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(j) a reduction in mean LDL particle number of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(k) an increase in mean LDL particle size of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(l) a reduction in remnant-like particle cholesterol of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(m) a reduction in oxidized LDL of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(n) substantially no change, no significant change, or a reduction (e.g. in the case of a diabetic subject) in fasting plasma glucose (FPG) of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(o) substantially no change, no significant change or a reduction in hemoglobin A_{1c} (HbA_{1c}) of at least about 5%, at least about 10%, at least about 15%, at least about 20%, at least about 25%, at least about 30%, at least about 35%, at least about 40%, at least about 45%, or at least about 50% (actual % change or median % change) compared to baseline;

(p) a reduction in homeostasis model index insulin resistance of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(q) a reduction in lipoprotein associated phospholipase A2 of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(r) a reduction in intracellular adhesion molecule-1 of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(s) a reduction in interleukin-6 of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(t) a reduction in plasminogen activator inhibitor-1 of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(u) a reduction in high sensitivity C-reactive protein (hsCRP) of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(v) an increase in serum, plasma and/or RBC EPA of at least about 5%, at least about 10%, at least about 15%, at least about 20%, at least about 25%, at least about 30%, at least about 35%, at least about 40%, at least about 45%, at least about 50%, at least about 100%, at least about 200%, at least about 250%, at least about 275%, 297.7%, about 298%, at least about 300%, at least about 400%, about 402%, 402.3%, at least about 450%, 489.6%, about 490%, at least about 500%, at least about 600%, at least about 700%, about 790%, 792%, or at least about 800% (actual % change or median % change) compared to baseline or placebo control;

(w) an increase in serum phospholipid and/or red blood cell membrane EPA of at least about 5%, at least about 10%, at least about 15%, at least about 20%, at least about 25%, at least about 30%, at least about 35%, at least about 40%, at least about 45%, r at least about 50%, at least about 100%, at least about 200%, or at least about 400% (actual % change or median % change) compared to baseline;

(x) a reduction or increase in one or more of serum phospholipid and/or red blood cell DHA, DPA, AA, PA and/or OA of at least about 5%, at least about 10%, at least about 15%, at least about 20%, at least about 25%, at least about 30%, at least about 35%, at least about 40%, at least about 45%, at least about 50%, at least about 55% or at least about 75% (actual % change or median % change) compared to baseline;

(y) a reduction in total cholesterol of at least about 5%, at least about 10%, at least about 15%, at least about 20%, at least about 25%, at least about 30%, at least about 35%, at least about 40%, at least about 45%, at least about 50%, at least about 55% or at least about 75% (actual % change or median % change) compared to baseline;

(z) a reduction in the ratio of arachidonic acid to EPA (“AA/EPA”) in serum, plasma, and/or RBCs of at least about 50%, at least about 75%, at least about 80%, at least about 85%, about 88%, 88.4%, about 91%, 91.1%, about 99%, 99.4%, at least about 100%, about 102%, 102%, at least about 125%, or at least about 150% (actual % change or median % change) compared to baseline or placebo control;

(aa) an increase in the ratio of omega-3 fatty acids to omega-6 fatty acids in serum, plasma, and/or RBCs of at least about 50%, at least about 60%, at least about 65%, about 65%, 65.5%, about 66%, at least about 70%, at least about 75%, 76.8%, about 77%, at least about 80%, at least about 90%, at least about 100%, 106.9%, about 107%, at least about 110%, at least about 120%, at least about 130%, at least about 140%, at least about 145%, 145.8%, about 146%, at least about 150%, at least about 160%, at least about 170%, at least about 180%, at least about 190%, or at least about 200% (actual % change or median % change) compared to baseline or placebo control;

(bb) a decrease in the ratio of omega-6 fatty acids to total fatty acids in serum, plasma, and/or RBCs of at least about 5%, at least about 10%, at least about 13%, about 13%, 13.2%, at least about 14%, at least about 15%, at least about 16%, at least about 17%, about 17%, 17.3%, at least about 18%, at least about 19%, or at least about 20% (actual % change or median % change) compared to baseline or placebo control; and/or

(cc) an increase in the ratio of omega-3 fatty acids to total fatty acids in serum, plasma, and/or RBCs of at least about 20%, 23.8%, about 24%, at least about 30%, at least about 40%, at least about 50%, about 53%, 53.4%, at least about 60%, about 68%, 68.4%, at least about 70%, at least about 80%, at least about 90%, at least about 100%, at least about 110%, at least about 120%, at least about 130%, 132.7%, about 133%, at least about 140%, at least about 150% (actual % change or median % change) compared to baseline or placebo control.

In one embodiment, methods of the present invention comprise measuring baseline levels of one or more markers set forth in (a)-(cc) prior to dosing the subject or subject group. In another embodiment, the methods comprise administering a composition as disclosed herein to the subject after baseline levels of one or more markers set forth in (a)-(cc) are determined, and subsequently taking a second measurement of the one or more markers as measured at baseline for comparison thereto.

In another embodiment, upon treatment with a composition of the present invention, for example over a period of about 1 to about 200 weeks, about 1 to about 100 weeks, about 1 to about 80 weeks, about 1 to about 50 weeks, about 1 to about 40 weeks, about 1 to about 20 weeks, about 1 to about 15 weeks, about 1 to about 12 weeks, about 1 to about 10 weeks, about 1 to about 5 weeks, about 1 to about 2 weeks or about 1 week, the subject or subject group exhibits any 2 or more of, any 3 or more of, any 4 or more of, any 5 or more of, any 6 or more of, any 7 or more of, any 8 or more of, any 9 or more of, any 10 or more of, any 11 or more of, any 12 or more of, any 13 or more of, any 14 or more of, any 15 or more of, any 16 or more of, any 17 or more of, any 18 or more of, any 19 or more of, any 20 or more of, any 21 or more of, any 22 or more of, any 23 or more of, any 24 or more of, any 25 or more of, any 26 or more of, any 27 or more of, any 28 or more of, or all 29 of outcomes (a)-(cc) described immediately above.

Parameters (a)-(cc) can be measured in accordance with any clinically acceptable methodology. For example, triglycerides, total cholesterol, HDL-C and fasting blood sugar can be sample from serum and analyzed using standard photometry techniques. VLDL-TG, LDL-C and VLDL-C can be calculated or determined using serum lipoprotein fractionation by preparative ultracentrifugation and subsequent quantitative analysis by refractometry or by analytic ultracentrifugal methodology. Apo A1, Apo B and hsCRP can be determined from serum using standard nephelometry techniques. Lipoprotein (a) can be determined from serum using standard turbidimetric immunoassay techniques. LDL particle number and particle size can be determined using nuclear magnetic resonance (NMR) spectrometry. Remnants lipoproteins and LDL-phospholipase A2 can be determined from EDTA plasma or serum and serum, respectively, using enzymatic immunoseparation techniques. Oxidized LDL, intercellular adhesion molecule-1 and interleukin-6 levels can be determined from serum using standard enzyme immunoassay techniques. These techniques are described in detail in standard textbooks, for example Tietz Fundamentals of Clinical Chemistry, 6^{th} Ed. (Burtis, Ashwood and Borter Eds.), WB Saunders Company.

In one embodiment, subjects fast for up to 12 hours prior to blood sample collection, for example about 10 hours.

In another embodiment, the present invention provides a method of treating or preventing primary hypercholesterolemia and/or mixed dyslipidemia (Fredrickson Types IIa and IIb) in a patient in need thereof, comprising administering to the patient one or more compositions as disclosed herein. In a related embodiment, the present invention provides a method of reducing triglyceride levels in a subject or subjects when treatment with a statin or niacin extended-release monotherapy is considered inadequate (Frederickson type IV hyperlipidemia).

In another embodiment, the present invention provides a method of treating or preventing risk of recurrent nonfatal myocardial infarction in a patient with a history of myocardial infarction, comprising administering to the patient one or more compositions as disclosed herein.

In another embodiment, the present invention provides a method of slowing progression of or promoting regression of atherosclerotic disease in a patient in need thereof, comprising administering to a subject in need thereof one or more compositions as disclosed herein.

In another embodiment, the present invention provides a method of treating or preventing very high serum triglyceride levels (e.g. Types IV and V hyperlipidemia) in a patient in need thereof, comprising administering to the patient one or more compositions as disclosed herein.

In another embodiment, the present invention provides a method of treating subjects having very high serum triglyceride levels (e.g. greater than 1000 mg/dl or greater than 2000 mg/dl) and that are at risk of developing pancreatitis, comprising administering to the patient one or more compositions as disclosed herein.

In one embodiment, a composition of the invention is administered to a subject in an amount sufficient to provide a daily dose of eicosapentaenoic acid of about 1 mg to about 10,000 mg, 25 about 5000 mg, about 50 to about 3000 mg, about 75 mg to about 2500 mg, or about 100 mg to about 1000 mg, for example about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1025 mg, about 1050 mg, about 1075 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg, about 2525 mg, about 2550 mg, about 2575 mg, about 2600 mg, about 2625 mg, about 2650 mg, about 2675 mg, about 2700 mg, about 2725 mg, about 2750 mg, about 2775 mg, about 2800 mg, about 2825 mg, about 2850 mg, about 2875 mg, about 2900 mg, about 2925 mg, about 2950 mg, about 2975 mg, about 3000 mg, about 3025 mg, about 3050 mg, about 3075 mg, about 3100 mg, about 3125 mg, about 3150 mg, about 3175 mg, about 3200 mg, about 3225 mg, about 3250 mg, about 3275 mg, about 3300 mg, about 3325 mg, about 3350 mg, about 3375 mg, about 3400 mg, about 3425 mg, about 3450 mg, about 3475 mg, about 3500 mg, about 3525 mg, about 3550 mg, about 3575 mg, about 3600 mg, about 3625 mg, about 3650 mg, about 3675 mg, about 3700 mg, about 3725 mg, about 3750 mg, about 3775 mg, about 3800 mg, about 3825 mg, about 3850 mg, about 3875 mg, about 3900 mg, about 3925 mg, about 3950 mg, about 3975 mg, about 4000 mg, about 4025 mg, about 4050 mg, about 4075 mg, about 4100 mg, about 4125 mg, about 4150 mg, about 4175 mg, about 4200 mg, about 4225 mg, about 4250 mg, about 4275 mg, about 4300 mg, about 4325 mg, about 4350 mg, about 4375 mg, about 4400 mg, about 4425 mg, about 4450 mg, about 4475 mg, about 4500 mg, about 4525 mg, about 4550 mg, about 4575 mg, about 4600 mg, about 4625 mg, about 4650 mg, about 4675 mg, about 4700 mg, about 4725 mg, about 4750 mg, about 4775 mg, about 4800 mg, about 4825 mg, about 4850 mg, about 4875 mg, about 4900 mg, about 4925 mg, about 4950 mg, about 4975 mg, about 5000 mg, about 5500 mg, about 6000 mg, about 6500 mg, about 7000 mg, about 7500 mg, about 8000 mg, about 8500 mg, about 9000 mg, about 9500 mg, or about 10,000 mg.

In another embodiment, any of the methods disclosed herein are used in treatment or prevention of a subject or subjects that consume a traditional Western diet. In one embodiment, the methods of the invention include a step of identifying a subject as a Western diet consumer or prudent diet consumer and then treating the subject if the subject is deemed a Western diet consumer. The term “Western diet” herein refers generally to a typical diet consisting of, by percentage of total calories, about 45% to about 50% carbohydrate, about 35% to about 40% fat, and about 10% to about 15% protein. A Western diet may alternately or additionally be characterized by relatively high intakes of red and processed meats, sweets, refined grains, and desserts, for example more than 50%, more than 60% or more or 70% of total calories come from these sources.

In one embodiment, a composition for use in methods of the invention comprises eicosapentaenoic acid, or a pharmaceutically acceptable ester, derivative, conjugate or salt thereof, or mixtures of any of the foregoing, collectively referred to herein as “EPA.” The term “pharmaceutically acceptable” in the present context means that the substance in question does not produce unacceptable toxicity to the subject or interaction with other components of the composition.

In one embodiment, the EPA comprises all-cis eicosa-5,8,11,14,17-pentaenoic acid. In another embodiment, the EPA comprises an eicosapentaenoic acid ester. In another embodiment, the EPA comprises a C₁-C₅ alkyl ester of eicosapentaenoic acid. In another embodiment, the EPA comprises eicosapentaenoic acid ethyl ester, eicosapentaenoic acid methyl ester, eicosapentaenoic acid propyl ester, or eicosapentaenoic acid butyl ester. In another embodiment, the EPA comprises In one embodiment, the EPA comprises all-cis eicosa-5,8,11,14,17-pentaenoic acid ethyl ester.

In another embodiment, the EPA is in the form of ethyl-EPA, lithium EPA, mono-, di- or triglyceride EPA or any other ester or salt of EPA, or the free acid form of EPA. The EPA may also be in the form of a 2-substituted derivative or other derivative which slows down its rate of oxidation but does not otherwise change its biological action to any substantial degree.

In another embodiment, EPA is present in a composition useful in accordance with methods of the invention in an amount of about 50 mg to about 5000 mg, about 75 mg to about 2500 mg, or about 100 mg to about 1000 mg, for example about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1025 mg, about 1050 mg, about 1075 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg or about 2500 mg, 2525 mg, about 2550 mg, about 2575 mg, about 2600 mg, about 2625 mg, about 2650 mg, about 2675 mg, about 2700 mg, about 2725 mg, about 2750 mg, about 2775 mg, about 2800 mg, about 2825 mg, about 2850 mg, about 2875 mg, about 2900 mg, about 2925 mg, about 2950 mg, about 2975 mg, about 3000 mg, about 3025 mg, about 3050 mg, about 3075 mg, about 3100 mg, about 3125 mg, about 3150 mg, about 3175 mg, about 3200 mg, about 3225 mg, about 3250 mg, about 3275 mg, about 3300 mg, about 3325 mg, about 3350 mg, about 3375 mg, about 3400 mg, about 3425 mg, about 3450 mg, about 3475 mg, about 3500 mg, about 3525 mg, about 3550 mg, about 3575 mg, about 3600 mg, about 3625 mg, about 3650 mg, about 3675 mg, about 3700 mg, about 3725 mg, about 3750 mg, about 3775 mg, about 3800 mg, about 3825 mg, about 3850 mg, about 3875 mg, about 3900 mg, about 3925 mg, about 3950 mg, about 3975 mg, about 4000 mg, about 4025 mg, about 4050 mg, about 4075 mg, about 4100 mg, about 4125 mg, about 4150 mg, about 4175 mg, about 4200 mg, about 4225 mg, about 4250 mg, about 4275 mg, about 4300 mg, about 4325 mg, about 4350 mg, about 4375 mg, about 4400 mg, about 4425 mg, about 4450 mg, about 4475 mg, about 4500 mg, about 4525 mg, about 4550 mg, about 4575 mg, about 4600 mg, about 4625 mg, about 4650 mg, about 4675 mg, about 4700 mg, about 4725 mg, about 4750 mg, about 4775 mg, about 4800 mg, about 4825 mg, about 4850 mg, about 4875 mg, about 4900 mg, about 4925 mg, about 4950 mg, about 4975 mg, or about 5000 mg.

In another embodiment, a composition useful in accordance with the invention contains not more than about 10%, not more than about 9%, not more than about 8%, not more than about 7%, not more than about 6%, not more than about 5%, not more than about 4%, not more than about 3%, not more than about 2%, not more than about 1%, or not more than about 0.5%, by weight, docosahexaenoic acid (DHA), if any. In another embodiment, a composition of the invention contains substantially no docosahexaenoic acid. In still another embodiment, a composition useful in the present invention contains no docosahexaenoic acid and/or derivative thereof.

In another embodiment, EPA comprises at least 70%, at least 80%, at least 90%, at least 95%, at least 96%, at least 97%, at least 98%, at least 99%, or 100%, by weight, of all fatty acids present in a composition that is useful in methods of the present invention.

In one embodiment, a composition of the invention comprises ultra-pure EPA. The term “ultra-pure” as used herein with respect to EPA refers to a composition comprising at least 95% by weight EPA (as the term “EPA” is defined and exemplified herein). Ultra-pure EPA comprises at least 96% by weight EPA, at least 97% by weight EPA, or at least 98% by weight EPA, wherein the EPA is any form of EPA as set forth herein.

In another embodiment, a composition useful in accordance with methods of the invention contains less than 10%, less than 9%, less than 8%, less than 7%, less than 6%, less than 5%, less than 4%, less than 3%, less than 2%, less than 1%, less than 0.5% or less than 0.25%, by weight of the total composition or by weight of the total fatty acid content, of any fatty acid other than EPA. Illustrative examples of a “fatty acid other than EPA” include linolenic acid (LA), arachidonic acid (AA), docosahexaenoic acid (DHA), alpha-linolenic acid (ALA), stearadonic acid (STA), eicosatrienoic acid (ETA) and/or docosapentaenoic acid (DPA). In another embodiment, a composition useful in accordance with methods of the invention contains about 0.1% to about 4%, about 0.5% to about 3%, or about 1% to about 2%, by weight, of total fatty acids other than EPA and/or DHA.

In another embodiment, a composition useful in accordance with the invention has one or more of the following features: (a) eicosapentaenoic acid ethyl ester represents at least about 96%, at least about 97%, or at least about 98%, by weight, of all fatty acids present in the composition; (b) the composition contains not more than about 4%, not more than about 3%, or not more than about 2%, by weight, of total fatty acids other than eicosapentaenoic acid ethyl ester; (c) the composition contains not more than about 0.6%, not more than about 0.5%, or not more than about 0.4% of any individual fatty acid other than eicosapentaenoic acid ethyl ester; (d) the composition has a refractive index (20° C.) of about 1 to about 2, about 1.2 to about 1.8 or about 1.4 to about 1.5; (e) the composition has a specific gravity (20° C.) of about 0.8 to about 1.0, about 0.85 to about 0.95 or about 0.9 to about 0.92; (e) the composition contains not more than about 20 ppm, not more than about 15 ppm or not more than about 10 ppm heavy metals, (f) the composition contains not more than about 5 ppm, not more than about 4 ppm, not more than about 3 ppm, or not more than about 2 ppm arsenic, and/or (g) the composition has a peroxide value of not more than about 5 meq/kg, not more than about 4 meq/kg, not more than about 3 meq/kg, or not more than about 2 meq/kg.

In another embodiment, a composition useful in accordance with the invention comprises, consists of or consists essentially of at least 95% by weight ethyl eicosapentaenoate (EPA-E), about 0.2% to about 0.5% by weight ethyl octadecatetraenoate (ODTA-E), about 0.05% to about 0.25% by weight ethyl nonadecapentaenoate (NDPA-E), about 0.2% to about 0.45% by weight ethyl arachidonate (AA-E), about 0.3% to about 0.5% by weight ethyl eicosatetraenoate (ETA-E), and about 0.05% to about 0.32% ethyl heneicosapentaenoate (HPA-E). In another embodiment, the composition is present in a capsule shell.

In another embodiment, compositions useful in accordance with the invention comprise, consist essential of, or consist of at least 95%, 96% or 97%, by weight, ethyl eicosapentaenoate, about 0.2% to about 0.5% by weight ethyl octadecatetraenoate, about 0.05% to about 0.25% by weight ethyl nonadecapentaenoate, about 0.2% to about 0.45% by weight ethyl arachidonate, about 0.3% to about 0.5% by weight ethyl eicosatetraenoate, and about 0.05% to about 0.32% ethyl heneicosapentaenoate. Optionally, the composition contains not more than about 0.06%, about 0.05%, or about 0.04%, by weight, DHA or derivative thereof such as ethyl-DHA. In one embodiment the composition contains substantially no or no amount of DHA or derivative thereof such as ethyl-DHA. The composition further optionally comprises one or more antioxidants (e.g. tocopherol) or other impurities in an amount of not more than about 0.5% or not more than 0.05%. In another embodiment, the composition comprises about 0.05% to about 0.4%, for example about 0.2% by weight tocopherol. In another embodiment, about 500 mg to about 1 g of the composition is provided in a capsule shell.

In another embodiment, compositions useful in accordance with the invention comprise, consist essential of, or consist of at least 96% by weight ethyl eicosapentaenoate, about 0.22% to about 0.4% by weight ethyl octadecatetraenoate, about 0.075% to about 0.20% by weight ethyl nonadecapentaenoate, about 0.25% to about 0.40% by weight ethyl arachidonate, about 0.3% to about 0.4% by weight ethyl eicosatetraenoate and about 0.075% to about 0.25% ethyl heneicosapentaenoate. Optionally, the composition contains not more than about 0.06%, about 0.05%, or about 0.04%, by weight, DHA or derivative thereof such as ethyl-DHA. In one embodiment the composition contains substantially no or no amount of DHA or derivative thereof such as ethyl-DHA. The composition further optionally comprises one or more antioxidants (e.g. tocopherol) or other impurities in an amount of not more than about 0.5% or not more than 0.05%. In another embodiment, the composition comprises about 0.05% to about 0.4%, for example about 0.2% by weight tocopherol. In another embodiment, the invention provides a dosage form comprising about 500 mg to about 1 g of the foregoing composition in a capsule shell. In one embodiment, the dosage form is a gel or liquid capsule and is packaged in blister packages of about 1 to about 20 capsules per sheet.

In another embodiment, compositions useful in accordance with the invention comprise, consist essential of, or consist of at least 96%, 97% or 98%, by weight, ethyl eicosapentaenoate, about 0.25% to about 0.38% by weight ethyl octadecatetraenoate, about 0.10% to about 0.15% by weight ethyl nonadecapentaenoate, about 0.25% to about 0.35% by weight ethyl arachidonate, about 0.31% to about 0.38% by weight ethyl eicosatetraenoate, and about 0.08% to about 0.20% ethyl heneicosapentaenoate. Optionally, the composition contains not more than about 0.06%, about 0.05%, or about 0.04%, by weight, DHA or derivative thereof such as ethyl-DHA. In one embodiment the composition contains substantially no or no amount of DHA or derivative thereof such as ethyl-DHA. The composition further optionally comprises one or more antioxidants (e.g. tocopherol) or other impurities in an amount of not more than about 0.5% or not more than 0.05%. In another embodiment, the composition comprises about 0.05% to about 0.4%, for example about 0.2% by weight tocopherol. In another embodiment, the invention provides a dosage form comprising about 500 mg to about 1 g of the foregoing composition in a capsule shell.

In another embodiment, a composition as described herein is administered to a subject once or twice per day. In another embodiment, 1, 2, 3 or 4 capsules, each containing about 1 g of a composition as described herein, are administered to a subject daily. In another embodiment, 1 or 2 capsules, each containing about 1 g of a composition as described herein, are administered to the subject in the morning, for example between about 5 am and about 11 am, and 1 or 2 capsules, each containing about 1 g of a composition as described herein, are administered to the subject in the evening, for example between about 5 pm and about 11 pm.

In one embodiment, a subject being treated in accordance with methods of the invention is not otherwise on lipid-altering therapy, for example statin, fibrate, niacin and/or ezetimibe therapy.

In another embodiment, compositions useful in accordance with methods of the invention are orally deliverable. The terms “orally deliverable” or “oral administration” herein include any form of delivery of a therapeutic agent or a composition thereof to a subject wherein the agent or composition is placed in the mouth of the subject, whether or not the agent or composition is swallowed. Thus “oral administration” includes buccal and sublingual as well as esophageal administration. In one embodiment, the composition is present in a capsule, for example a soft gelatin capsule.

A composition for use in accordance with the invention can be formulated as one or more dosage units. The terms “dose unit” and “dosage unit” herein refer to a portion of a pharmaceutical composition that contains an amount of a therapeutic agent suitable for a single administration to provide a therapeutic effect. Such dosage units may be administered one to a plurality (i.e. 1 to about 10, 1 to 8, 1 to 6, 1 to 4 or 1 to 2) of times per day, or as many times as needed to elicit a therapeutic response.

In another embodiment, the invention provides use of any composition described herein for treating moderate to severe hypertriglyceridemia in a subject in need thereof, comprising: providing a subject having a fasting baseline triglyceride level of about 500 mg/dl to about 1500 mg/dl and administering to the subject a pharmaceutical composition as described herein. In one embodiment, the composition comprises about 1 g to about 4 g of eicosapentaenoic acid ethyl ester, wherein the composition contains substantially no docosahexaenoic acid.

In one embodiment, compositions of the invention, upon storage in a closed container maintained at room temperature, refrigerated (e.g. about 5 to about 5-10° C.) temperature, or frozen for a period of about 1, 2, 3, 4, 5, 6, 7, 8, 9, 10, 11, or 12 months, exhibit at least about 90%, at least about 95%, at least about 97.5%, or at least about 99% of the active ingredient(s) originally present therein.

In one embodiment, the invention provides use of a composition as described herein in manufacture of a medicament for treatment of any of a cardiovascular-related disease. In another embodiment, the subject is diabetic.

In one embodiment, a composition as set forth herein is packaged together with instructions for using the composition to treat a cardiovascular disorder.

### Examples

A multi-center, placebo-controlled randomized, double-blind, 12-week study with an open-label extension is performed to evaluate the efficacy and safety of AMR101 in patients with fasting triglyceride levels ≧500 mg/dL. The primary objective of the study is to determine the efficacy of AMR101 2 g daily and 4 g daily, compared to placebo, in lowering fasting TG levels in patients with fasting TG levels ≧500 mg/dL and ≦1500 mg/dL (≧5.65 mmol/L and ≦16.94 mmol/L).

The secondary objectives of this study are the following:
1. To determine the safety and tolerability of AMR101 2 g daily and 4 g daily;
2. To determine the effect of AMR101 on lipid and apolipoprotein profiles;
3. To determine the effect of AMR101 on low-density lipoprotein (LDL) particle number and size;
4. To determine the effect of AMR101 on oxidized LDL;
5. To determine the effect of AMR101 on fasting plasma glucose (FPG) and hemoglobin A_{1c} (HbA_{1c});
6. To determine the effect of AMR101 on insulin resistance;
7. To determine the effect of AMR101 on high-sensitivity C-reactive protein (hsCRP);
8. To determine the effects of AMR101 2 g daily and 4 g daily on the incorporation of fatty acids into red blood cell membranes and into plasma phospholipids;
9. To explore the relationship between baseline fasting TG levels and the reduction in fasting TG levels; and
10. To explore the relationship between an increase in red blood cell membrane eicosapentaenoic acid (EPA) concentrations and the reduction in fasting TG levels.

The population for this study is men and women (women of childbearing potential will need to be on contraception or practice abstinence) >18 years of age with a body mass index ≦45 kg/m² who are not on lipid-altering therapy or are currently on lipid-altering therapy. Patients currently on statin therapy (with or without ezetimibe) will be evaluated by the investigator as to whether this therapy can be safely discontinued at screening, or if it should be continued. If statin therapy (with or without ezetimibe) is to be continued, dose(s) must be stable for ≧4 weeks prior to randomization. Patients taking non-statin, lipid-altering medications (niacin >200 mg/day, fibrates, fish oil, other products containing omega-3 fatty acids, or other herbal products or dietary supplements with potential lipid-altering effects), either alone or in combination with statin therapy (with or without ezetimibe), must be able to safely discontinue non-statin, lipid-altering therapy at screening.

Approximately 240 patients will be randomized at approximately 50 centers in North America, South America, Central America, Europe, India, and South Africa. The study will be a 58- to 60-week, Phase 3, multi-center study consisting of 3 study periods: (1) A 6- to 8-week screening period that includes a diet and lifestyle stabilization and washout period and a TG qualifying period; (2) A 12-week, double-blind, randomized, placebo-controlled treatment period; and (3) A 40-week, open-label, extension period.

During the screening period and double-blind treatment period, all visits are to be within ±3 days of the scheduled time. During the open-label extension period, all visits are to be within ±7 days of the scheduled time. The screening period includes a 4- or 6-week diet and lifestyle stabilization period and washout period followed by a 2-week TG qualifying period. s) must be stable for ≧4 weeks prior to randomization.

The screening visit (Visit 1) will occur for all patients at either 6 weeks (for patients not on lipid-altering therapy at screening or for patients who will not need to discontinue their current lipid-altering therapy) or 8 weeks (for patients who will require washout of their current lipid-altering therapy at screening) before randomization, as follows:

Patients who do not require a washout: The screening visit will occur at Visit 1 (Week-6). Eligible patients will enter a 4-week diet and lifestyle stabilization period. At the screening visit, all patients will receive counseling regarding the importance of the National Cholesterol Education Program (NCEP) Therapeutic Lifestyle Changes (TLC) diet and will receive instructions on how to follow this diet. Patients who will require a washout: The screening visit will occur at Visit 1 (Week-8). Eligible patients will begin a 6-week washout period at the screening visit. Patients will receive counseling regarding the NCEP TLC diet and will receive instructions on how to follow this diet. Site personnel will contact patients who do not qualify for participation based on screening laboratory test results to instruct them to resume their prior lipid-altering medications.

At the end of the 4-week diet and lifestyle stabilization period or the 6-week diet and stabilization and washout period, eligible patients will enter the 2-week TG qualifying period and will have their fasting TG level measured at Visit 2 (Week-2) and Visit 3 (Week-1). Eligible patients must have an average fasting TG level ≧500 mg/dL and ≦1500 mg/dL (≧5.65 mmol/L and ≦16.94 mmol/L) to enter the 12-week double-blind treatment period. The TG level for qualification will be based on the average (arithmetic mean) of the Visit 2 (Week-2) and Visit 3 (Week-1) values. If a patient's average TG level from Visit 2 and Visit 3 falls outside the required range for entry into the study, an additional sample for fasting TG measurement can be collected 1 week later at Visit 3.1. If a third sample is collected at Visit 3.1, entry into the study will be based on the average (arithmetic mean) of the values from Visit 3 and Visit 3.1.

After confirmation of qualifying fasting TG values, eligible patients will enter a 12-week, randomized, double-blind treatment period. At Visit 4 (Week 0), patients will be randomly assigned to 1 of the following treatment groups:

AMR101 2 g daily,

AMR101 4 g daily, or

Placebo.

During the double-blind treatment period, patients will return to the site at Visit 5 (Week 4), Visit 6 (Week 11), and Visit 7 (Week 12) for efficacy and safety evaluations.

Patients who complete the 12-week double-blind treatment period will be eligible to enter a 40-week, open-label, extension period at Visit 7 (Week 12). All patients will receive open-label AMR101 4 g daily. From Visit 8 (Week 16) until the end of the study, changes to the lipid-altering regimen are permitted (e.g., initiating or raising the dose of statin or adding non-statin, lipid-altering medications to the regimen), as guided by standard practice and prescribing information. After Visit 8 (Week 16), patients will return to the site every 12 weeks until the last visit at Visit 11 (Week 52).

Eligible patients will be randomly assigned at Visit 4 (Week 0) to receive orally AMR101 2 g daily, AMR101 4 g daily, or placebo for the 12-week double-blind treatment period. AMR101 is provided in 1 g liquid-filled, oblong, gelatin capsules. The matching placebo capsule is filled with light liquid paraffin and contains 0 g of AMR101. During the double-blind treatment period, patients will take 2 capsules (AMR101 or matching placebo) in the morning and 2 in the evening for a total of 4 capsules per day. Patients in the AMR101 2 g/day treatment group will receive 1 AMR101 1 g capsule and 1 matching placebo capsule in the morning and in the evening. Patients in the AMR101 4 g/day treatment group will receive 2 AMR101 1 g capsules in the morning and evening.

Patients in the placebo group will receive 2 matching placebo capsules in the morning and evening. During the extension period, patients will receive open-label AMR101 4 g daily. Patients will take 2 AMR101 1 g capsules in the morning and 2 in the evening.

The primary efficacy variable for the double-blind treatment period is percent change in TG from baseline to Week 12 endpoint. The secondary efficacy variables for the double-blind treatment period include the following:
Percent changes in total cholesterol (TC), high-density lipoprotein cholesterol (HDL-C), calculated low-density lipoprotein cholesterol (LDL-C), calculated non-high-density lipoprotein cholesterol (non-HDL-C), and very low-density lipoprotein cholesterol (VLDL-C) from baseline to Week 12 endpoint;
Percent change in very low-density lipoprotein TG from baseline to Week 12;
Percent changes in apolipoprotein A-I (apo A-I), apolipoprotein B (apo B), and apo A-I/apo B ratio from baseline to Week 12;
Percent changes in lipoprotein (a) from baseline to Week 12 (selected sites only);
Percent changes in LDL particle number and size, measured by nuclear magnetic resonance, from baseline to Week 12 (selected sites only);
Percent change in remnant-like particle cholesterol from baseline to Week 12 (selected sites only);
Percent change in oxidized LDL from baseline to Week 12 (selected sites only);
Changes in FPG and HbA_{1c} from baseline to Week 12;
Change in insulin resistance, as assessed by the homeostasis model index insulin resistance, from baseline to Week 12;
Percent change in lipoprotein associated phospholipase A2 from baseline to Week 12 (selected sites only);
Change in intracellular adhesion molecule-1 from baseline to Week 12 (selected sites only);
Change in interleukin-6 from baseline to Week 12 (selected sites only);
Change in plasminogen activator inhibitor-1 from baseline to Week 12 (selected sites only);
Change in hsCRP from baseline to Week 12 (selected sites only);
Change in serum phospholipid EPA content from baseline to Week 12;
Change in red blood cell membrane EPA content from baseline to Week 12; and
Change in serum phospholipid and red blood cell membrane content in the following fatty acids from baseline to Week 12: docosapentaenoic acid, docosahexaenoic acid, arachidonic acid, palmitic acid, stearic acid, and oleic acid.

The efficacy variable for the open-label extension period is percent change in fasting TG from extension baseline to end of treatment. Safety assessments will include adverse events, clinical laboratory measurements (chemistry, hematology, and urinalysis), 12-lead electrocardiograms (ECGs), vital signs, and physical examinations

For TG, TC, HDL-C, calculated LDL-C, calculated non-HDL-C, and VLDL-C, baseline will be defined as the average of Visit 4 (Week 0) and the preceding lipid qualifying visit (either Visit 3 [Week-1] or if it occurs, Visit 3.1) measurements. Baseline for all other efficacy parameters will be the Visit 4 (Week 0) measurement.

For TC, HDL-C, calculated LDL-C, calculated non-HDL-C, and VLDL-C, Week 12 endpoint will be defined as the average of Visit 6 (Week 11) and Visit 7 (Week 12) measurements. Week 12 endpoint for all other efficacy parameters will be the Visit 7 (Week 12) measurement.

The primary efficacy analysis will be performed using a 2-way analysis of covariance (ANCOVA) model with treatment as a factor and baseline TG value as a covariate. The least-squares mean, standard error, and 2-tailed 95% confidence interval for each treatment group and for each comparison will be estimated. The same 2-way ANCOVA model will be used for the analysis of secondary efficacy variables.

The primary analysis will be repeated for the per-protocol population to confirm the robustness of the results for the intent-to-treat population.

The primary efficacy variable will be the percent change in fasting TG levels from baseline to Week 12. A sample size of 69 completed patients per treatment group will provide ≧90% power to detect a difference of 30% between AMR101 and placebo in percent change from baseline in fasting TG levels, assuming a standard deviation of 45% in TG measurements and a significance level of p<0.01. To accommodate a 15% drop-out rate from randomization to completion of the double-blind treatment period, a total of 240 randomized patients is planned (80 patients per treatment group).

### Results

Of the 229 randomized subjects, 224 were in the intent-to-treat (“ITT”) population as follows:
Ultra-pure EPA, 4 g/day: 76 subjects
Ultra-pure EPA, 2 g/day: 73 subjects
Placebo: 75 subjects

Lipids were extracted from plasma and RBC suspensions and converted into fatty acid methyl esters for analysis using a standard validated gas chromatography/flame ionization detection method. Fatty acid parameters were compared between EPA treatment groups and placebo using an ANCOVA model with treatment, gender, type of statin therapy, and presence of diabetes as factors, and the baseline parameter value as a covariate. LSMs, SEs, and 2-tailed 95% confidence intervals for each treatment group and for each comparison were determined.

Baseline characteristics of the three ITT groups were comparable, with 76% of the ITT subjects being male, 88% being white, having a mean age of 52.9 years, a weight of 95.7 kg and a BMI of 30.8 kg/m². The median triglyceride level of the ITT subjects was 679.5 mg/dl. ITT subjects with incomplete fatty acid data at baseline and/or at 12 weeks were excluded from the analyses described below (as reflected in the n numbers shown in Tables 1-3).

As shown in Table 1, ultra-pure EPA at 2 g/day and 4 g/day both significantly increased mean placebo-adjusted plasma concentrations of EPA, docosapentaenoic acid m-3 (“DPAn-3”), and the ratio of total omega-3 to omega-6 fatty acids (“Ratio of n-3/n-6 FA”). In addition, both 2 g/day and 4 g/day doses of ultra-pure EPA significantly decreased mean placebo-adjusted plasma concentrations of arachidonic acid, palmitic acid, stearic acid, oleic acid, linoleic acid, and the plasma ratio of arachidonic acid to EPA. Neither dose of ultra-pure EPA produced a significant change in mean placebo-adjusted plasma concentration of DHA.

As shown in Table 2, ultra-pure EPA at 2 g/day and 4 g/day both significantly increased mean placebo-adjusted concentrations of EPA, DPAn-3, and the ratio of total omega-3 to omega-6 fatty acids (“Ratio of n-3/n-6 FA”) in RBCs. In addition, both 2 g/day and 4 g/day doses of ultra-pure EPA significantly decreased mean placebo-adjusted RBC concentrations of arachidonic acid and the RBC ratio of arachidonic acid to EPA. The RBC concentration of linoleic acid significantly decreased over placebo for subjects in the 4 g/day EPA group. Neither dose of ultra-pure EPA produced a significant change in mean placebo-adjusted RBC concentrations of DHA.

Table 3 shows changes from baseline and placebo-adjusted percent changes from baseline in the proportion (mol %) of fatty acid classes to total fatty acids in both plasma and in RBCs. The monounsaturated fatty acid group included myristoleic, palmitoleic, cis-vaccenic, oleic, gondoic/gadoleic, erucic and nervonic acids. The saturated fatty acid group included myristic, palmitic, stearic, arachidic, behenic and lignoceric acids. The “total omega-3” group included α-linolenic, stearidonic, eicosatrienoic, ω-3 arachidonic, eicosapentaenoic, ω-3 docosapentaenoic and docosahecaenoic acids. The “total omega-6” group included linoleic, γ-linolenic, eicosadienoic, dihomo-γ-linolenic, ω-6 arachidonic, adrenic, and ω-6 docosapentaenoic acids. The “total fatty acid” group represented the sum of the monounsaturated, saturated, total omega-3 and total omega-6 groups. Treatment with 2 g/day or 4 g/day ultra-pure EPA resulted in an increased proportion of omega-3 fatty acids in plasma and in RBCs compared to placebo, and a decreased proportion of omega-6 fatty acids in plasma compared to placebo. Treatment with 2 g/day or 4 g/day ultra-pure EPA also resulted in a decreased proportion of monounsaturated fatty acids in plasma and RBCs compared to placebo. Neither dose induced significant changes in the proportion of saturated fatty acids in plasma or in RBCs.

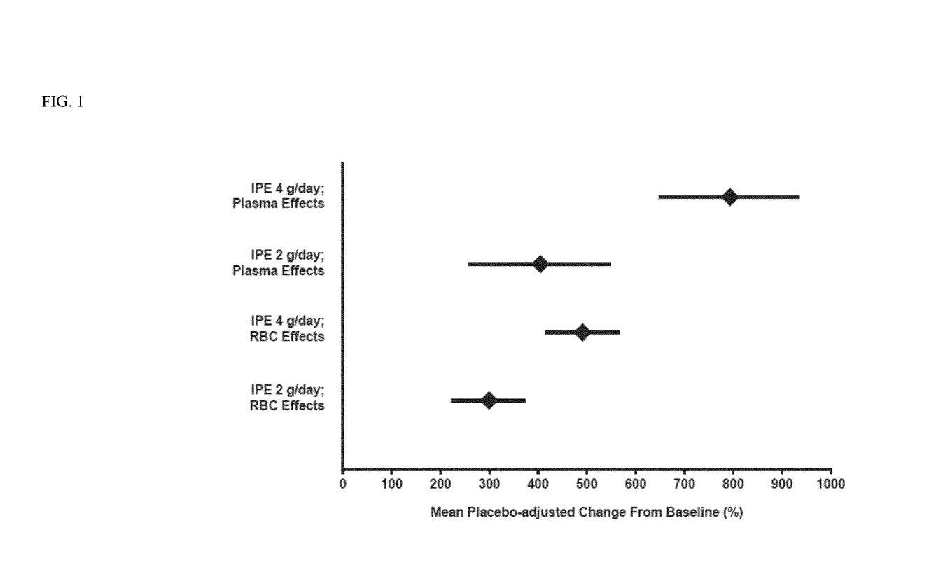

FIG. 1 shows the placebo-adjusted least squares mean changes (P<0.0001) in EPA concentration from baseline to week 12 in plasma and RBCs. The error bars represent 95% confidence intervals. Treatment with 4 g/day ultra-pure EPA (referred to as “IPE” in the Figures) resulted in a 792.0% placebo-adjusted LS mean increase in plasma EPA over baseline, and a 489.6% placebo-adjusted LS mean increase in RBC EPA over baseline. Treatment with 2 g/day ultra-pure EPA resulted in a 402.3% placebo-adjusted LS mean increase in plasma EPA over baseline, and a 297.7% placebo-adjusted LS mean increase in RBC EPA over baseline.

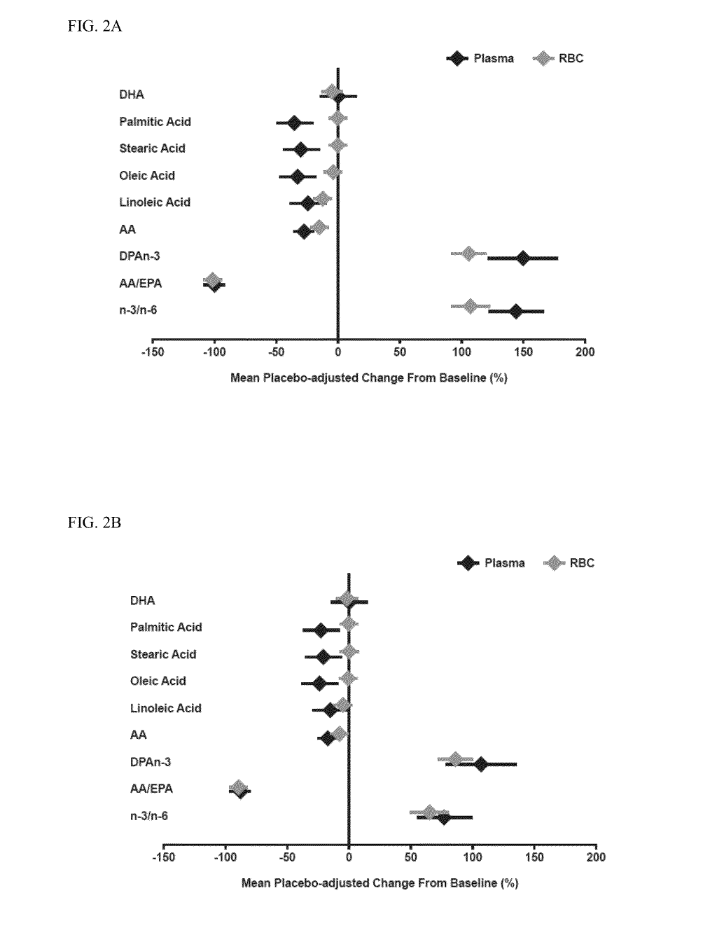

FIG. 2A shows plasma (dark diamonds) and RBC (light diamonds) placebo-adjusted least square mean changes from baseline to week 12 for select individual fatty acids and ratios of arachidonic acid to EPA (“AA/EPA”) and total omega-3 fatty acids to total omega-6 fatty acids (“n-3/n-6”) upon treatment with 4 g/day ultra-pure EPA. The values for each data point and corresponding P values are reported in Tables 1 and 2. Error bars represent 95% confidence intervals.

FIG. 2B shows plasma (dark diamonds) and RBC (light diamonds) placebo-adjusted least square mean changes from baseline to week 12 for select individual fatty acids and ratios of arachidonic acid to EPA (“AA/EPA”) and total omega-3 fatty acids to total omega-6 fatty acids (“n-3/n-6”) upon treatment with 2 g/day ultra-pure EPA. The values for each data point and corresponding P values are reported in Tables 1 and 2. Error bars represent 95% confidence intervals.

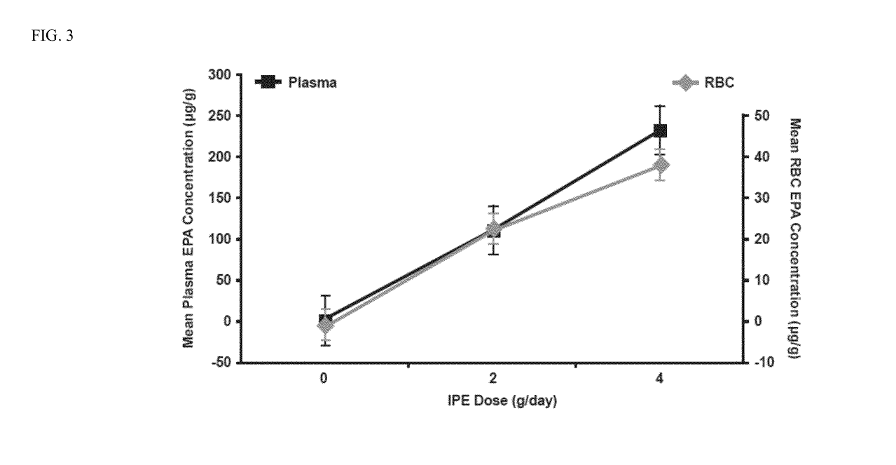

FIG. 3 depicts the ultra-pure EPA dose dependence of changes in EPA plasma and RBC concentration from baseline. Increased doses of ultra-pure EPA resulted in increased EPA concentration in both plasma (dark squares, y-axis on left) and RBCs (light diamonds, y-axis on right). Error bars represent 95% confidence intervals for each data point.

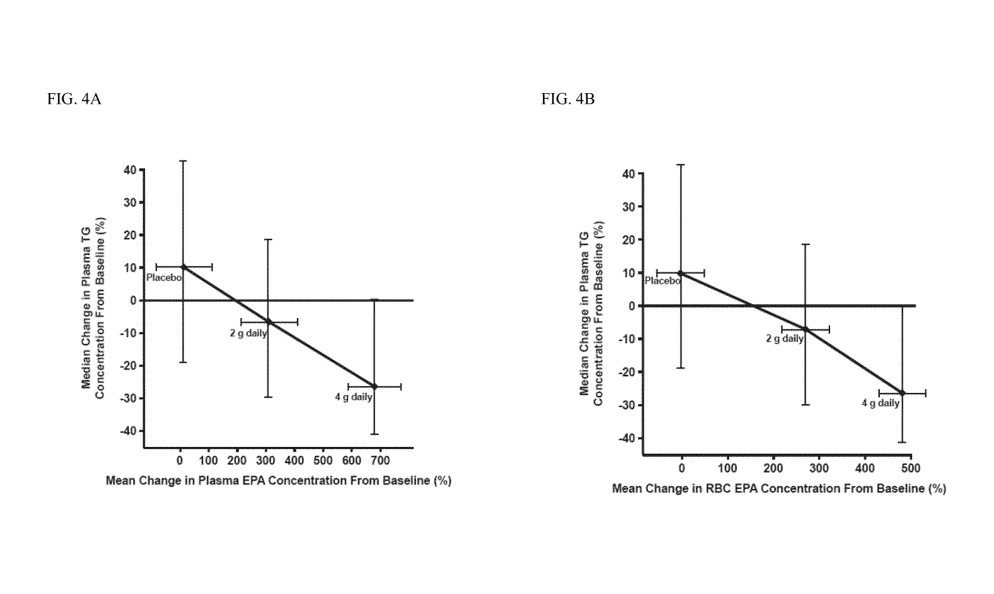

FIG. 4 shows the relationship between plasma triglyceride lowering and changes in EPA plasma and RBC concentrations (a pharmacokinetic/pharmacodynamic relationship). Increased EPA concentrations in plasma (FIG. 4A) and RBCs (FIG. 4B) were accompanied by greater reductions in plasma triglyceride levels. Triglyceride concentrations are presented in FIG. 4 as median percent changes from baseline. Vertical error bars represent the first and third quartile of the IQR. EPA concentrations are presented in FIG. 4 as mean percent changes from baseline. Horizontal error bars represent 95% confidence intervals.

These data indicate that ultra-pure EPA at either 2 g/day or 4 g/day significantly increased the concentrations of EPA and its metabolite, DPAn-3, in plasma and RBCs; significantly reduced the arachidonic acid/EPA ratio in plasma and RBCs (suggested by others to be a useful biomarker for arteriosclerotic disease); decreased the concentrations of key fatty acids including arachidonic, palmitic, stearic, oleic and linoleic acids in plasma and arachidonic acid (both doses) and linoleic acid (4 g/day only) in RBCs; increased the proportion of omega-3 fatty acids and decreased the proportion of omega-6 fatty acids in plasma and RBCs; and did not significantly increase the concentration of DHA in plasma or RBCs, indicating that its metabolic effects (including triglyceride lowering) are not due to increases in DHA levels.

Overall, administration of 2 g/day or 4 g/day ultra-pure EPA significantly increased EPA concentrations in a linear, dose-dependent fashion consistent with its triglyceride-lowering effect, increased the concentrations of docosapentaenoic acid n-3 (an EPA metabolite), caused beneficial shifts in the fatty acid profile, and significantly decreased the arachidonic acid/EPA ratio in plasma and RBCs.

TABLE 1
Icosapent ethyl (IPE): change from baseline to week 12 in plasma fatty acid concentrations and ratios.

| | IPE 4 g/day (n = 76) | | | IPE 2 g/day (n = 73) | | |
|---|---|---|---|---|---|---|
| | | End-of- | Change from | | End-of- | Change from |
| | Baseline | treatment | baseline | Baseline | treatment | baseline |
| | (SD) | (SD) | (SE) | (SD) | (SD) | (SE) |
| EPA | 50.4 | 279.8 | 233.0 (15.1) | 50.0 | 157.5 | 111.8 (14.4) |
| n = 48, 53, 44 | (52.0) | (161.3) | | (45.5) | (71.0) | |
| DHA | 125.5 | 118.1 | −4.5 (6.6) | 118.2 | 104.8 | −13.1 (6.2) |
| n = 48, 55, 46 | (93.1) | (81.6) | | (67.1) | (43.5) | |
| DPAn-3 | 37.5 | 91.1 | 54.6 (4.3) | 38.0 | 73.4 | 36.7 (3.9) |
| n = 46, 56, 47 | (20.8) | (43.7) | | (19.1) | (26.0) | |
| AA | 359.0 | 280.3 | −76.2 (11.3) | 368.6 | 327.3 | −36.7 (10.6) |
| n = 47, 56, 48 | (119.6) | (93.9) | | (121.4) | (101.4) | |
| Palmitic | 2287 | 1789 | −560 (138.1) | 2297 | 2015 | −353 (126.9) |
| n = 44, 54, 48 | (1344.1) | (1006.9) | | (1070.9) | (661.3) | |
| Stearic | 599.5 | 500.3 | −101 (35.4) | 577.9 | 541.5 | −57.8 (33.3) |
| n = 47, 55, 47 | (317.6) | (239.7) | | (229.7) | (165.2) | |
| Oleic | 2353 | 1885 | −546 (154.0) | 2322 | 2018 | −414 (142.9) |
| n = 46, 55, 46 | (1499.9) | (1130.3) | | (1246.1) | (837.7) | |
| Linoleic | 2098 | 1882 | −309 (128.1) | 2113 | 1981 | −227 (121.4) |
| n = 47, 54, 48 | (872.3) | (910.8) | | (934.8) | (775.1) | |
| Ratio of | 12.3 | 1.2 | −10.8 (0.5) | 11.4 | 2.3 | −9.6 (0.5) |
| AA/EPA | (7.9) | (0.9) | | (6.0) | (1.4) | |
| n = 45, 53, 41 | | | | | | |
| Ratio of | 0.11 | 0.24 | 0.4 (0.009) | 0.11 | 0.17 | 0.07 (0.009) |
| n-3/n-6 FA | (0.050) | (0.090) | | (0.062) | (0.071) | |
| n = 49, 55, 46 | | | | | | |

| | | | | Mean placebo-adjusted | |
|---|---|---|---|---|---|
| | | | | percent change from | |
| | Placebo (n = 75) | | | baseline | |
| | | End-of- | Change from | IPE 4 g/day | IPE 2 g/day |
| | Baseline | treatment | baseline | vs placebo, | vs placebo, |
| | (SD) | (SD) | (SE) | %, P | %, P |
| EPA | 41.0 | 37.4 | 1.4 (15.8) | 792.0 | 402.3 |
| n = 48, 53, 44 | (22.4) | (24.3) | | <0.0001 | <0.0001 |
| DHA | 112.2 | 113.4 | −0.7 (6.8) | 2.4 | −2.3 |
| n = 48, 55, 46 | (56.5) | (63.6) | | 0.7115 | 0.7217 |
| DPAn-3 | 36.7 | 39.4 | 4.1 (4.3) | 150.8 | 107.2 |
| n = 46, 56, 47 | (13.2) | (15.6) | | <0.0001 | <0.0001 |
| AA | 338.7 | 361.0 | 17.8 (11.3) | −26.8 | −16.4 |
| n = 47, 56, 48 | (95.7) | (121.4) | | <0.0001 | <0.0001 |
| Palmitic | 2418 | 2687 | 252.1 (135.4) | −34.4 | −23.9 |
| n = 44, 54, 48 | (1107.2) | (1306.6) | | <0.0001 | 0.0011 |
| Stearic | 646.4 | 709.9 | 79.6 (36.0) | −28.4 | −20.7 |
| n = 47, 55, 47 | (306.5) | (359.0) | | 0.0002 | 0.0040 |
| Oleic | 2653 | 2878 | 274.9 (156.7) | −31.9 | −23.8 |
| n = 46, 55, 46 | (1460.3) | (1616.0) | | 0.0001 | 0.0023 |
| Linoleic | 2374 | 2521 | 167.3 (129.0) | −23.7 | −15.2 |
| n = 47, 54, 48 | (1178.7) | (1174.6) | | 0.0003 | 0.0136 |
| Ratio of | 11.1 | 12.2 | 0.4 (0.5) | −99.4 | −88.4 |
| AA/EPA | (5.7) | (6.0) | | <0.0001 | <0.0001 |
| n = 45, 53, 41 | | | | | |
| Ratio of | 0.10 | 0.10 | 0.00 (0.010) | 145.8 | 76.8 |
| n-3/n-6 FA | (0.036) | (0.044) | | <0.0001 | <0.0001 |
| n = 49, 55, 46 | | | | | |

Table 1 Notes:
Mean and standard deviation are reported for baseline and end-of-treatment values; least squares mean and standard error are reported for changes from baseline and placebo-adjusted changes from baseline.
Abbreviations:
AA = arachidonic acid;
DHA = docosahexaenoic acid;
DPAn-3 = docosahexaenoic acid (omega-3);
EPA = eicosapentaenoic acid;
n-3/n-6 FA = ratio of total omega-3 fatty acids to total omega-6 fatty acids.

TABLE 2
Icosapent ethyl (IPE): change from baseline
to week 12 in red blood cell fatty acid concentrations and ratios.

| | IPE 4 g/day (n = 76) | | | IPE 2 g/day (n = 73) | | |
|---|---|---|---|---|---|---|
| | | End-of- | Change from | | End-of- | Change from |
| | Baseline | treatment | baseline | Baseline | treatment | baseline |
| | (SD) | (SD) | (SE) | (SD) | (SD) | (SE) |
| EPA | 11.4 | 50.2 | 39.4 (2.0) | 10.1 | 32.8 | 23.3 (1.9) |
| n = 50, 56, 45 | (6.9) | (18.0) | | (5.6) | (14.7) | |
| DHA | 62.1 | 51.7 | −9.7 (1.6) | 58.3 | 51.5 | −7.3 (1.5) |
| n = 48, 52, 43 | (20.0) | (16.6) | | (17.1) | (14.6) | |
| DPAn-3 | 28.9 | 56.9 | 27.7 (1.5) | 28.2 | 49.8 | 21.6 (1.4) |
| n = 48, 53, 40 | (5.7) | (14.3) | | (6.9) | (12.3) | |
| AA | 186.1 | 148.8 | −36.2 (3.4) | 179.9 | 160.3 | −20.9 (3.2) |
| n = 47, 53, 44 | (34.6) | (25.2) | | (34.7) | (33.5) | |
| Palmitic | 346.1 | 340.6 | −5.8 (5.7) | 344.1 | 341.7 | −2.9 (5.4) |
| n = 48, 53, 44 | (44.5) | (42.6) | | (45.1) | (51.9) | |
| Stearic | 186.5 | 178.1 | −8.2 (3.1) | 181.1 | 179.0 | −4.7 (2.9) |
| n = 50, 55, 46 | (30.6) | (24.8) | | (25.7) | (25.0) | |
| Oleic | 215.5 | 208.3 | −5.6 (4.1) | 216.0 | 213.0 | −1.4 (4.1) |
| n = 48, 50, 42 | (45.0) | (37.1) | | (44.5) | (39.5) | |
| Linoleic | 188.7 | 164.7 | −25.0 (4.9) | 176.3 | 169.6 | −10.7 (4.7) |
| n = 46, 53, 43 | (40.6) | (40.8) | | (38.5) | (42.5) | |
| Ratio of | 22.6 | 3.1 | −19.9 (1.0) | 22.2 | 5.2 | −17.7 (0.9) |
| AA/EPA | (12.4) | (1.6) | | (10.5) | (2.0) | |
| n = 48, 54, 44 | | | | | | |
| Ratio of | 0.22 | 0.42 | 0.20 (0.011) | 0.22 | 0.33 | 0.12 (0.010) |
| n-3/n-6 FA | (0.072) | (0.114) | | (0.068) | (0.102) | |
| n = 50, 56, 43 | | | | | | |

| | | | | Mean placebo-adjusted | |
|---|---|---|---|---|---|
| | | | | percent change from | |
| | Placebo (n = 75) | | | baseline | |
| | | End-of- | Change from | IPE 4 g/day | IPE 2 g/day |
| | Baseline | treatment | baseline | vs placebo, | vs placebo, |
| | (SD) | (SD) | (SE) | %, P | %, P |
| EPA | 10.1 | 8.2 | −1.1 (2.1) | 489.6 | 297.7 |
| n = 50, 56, 45 | (5.7) | (5.2) | | <0.0001 | <0.0001 |
| DHA | 60.1 | 54.4 | −5.6 (1.7) | −5.2 | −1.8 |
| n = 48, 52, 43 | (21.3) | (19.6) | | 0.1216 | 0.5859 |
| DPAn-3 | 29.0 | 27.0 | −2.4 (1.6) | 105.6 | 86.4 |
| n = 48, 53, 40 | (4.5) | (4.8) | | <0.0001 | <0.0001 |
| AA | 176.0 | 171.4 | −7.6 (3.5) | −15.7 | −8.0 |
| n = 47, 53, 44 | (27.2) | (25.5) | | <0.0001 | 0.0008 |
| Palmitic | 341.5 | 341.2 | −1.4 (6.0) | −0.7 | −0.1 |
| n = 48, 53, 44 | (37.7) | (53.3) | | 0.7427 | 0.9465 |
| Stearic | 179.8 | 176.6 | −6.5 (3.2) | −0.7 | 1.1 |
| n = 50, 55, 46 | (23.9) | (21.4) | | 0.7565 | 0.5886 |
| Oleic | 210.3 | 214.7 | 4.8 (4.4) | −3.7 | −2.1 |
| n = 48, 50, 42 | (35.3) | (46.3) | | 0.1325 | 0.3736 |
| Linoleic | 186.6 | 187.6 | −0.9 (5.1) | −13.0 | −4.7 |
| n = 46, 53, 43 | (32.4) | (42.3) | | <0.0001 | 0.1278 |
| Ratio of | 24.1 | 25.7 | 2.4 (1.0) | −102 | −91.1 |
| AA/EPA | (15.4) | (13.3) | | <0.0001 | <0.0001 |
| n = 48, 54, 44 | | | | | |
| Ratio of | 0.22 | 0.20 | −0.01 (0.012) | 106.9 | 65.5 |
| n-3/n-6 FA | (0.076) | (0.072) | | <0.0001 | <0.0001 |
| n = 50, 56, 43 | | | | | |

Table 2 Notes:
Mean and standard deviation are reported for baseline and end-of-treatment values; least-squares mean and standard error are reported for changes from baseline and placebo-adjusted changes from baseline.
Abbreviations:
AA = arachidonic acid;
DHA = docosahexaenoic acid;
DPAn-3 = docosahexaenoic acid (omega-3);
EPA = eicosapentaenoic acid;
n-3/n-6 FA = ratio of total omega-3 fatty acids to total omega-6 fatty acids.

TABLE 3
Icosapent ethyl: placebo-adjusted percent change in the proportion of fatty acid classes to total^{a} fatty acids.

| | | | | | | | | | Mean placebo-adjusted | |
|---|---|---|---|---|---|---|---|---|---|---|
| | | | | | | | | | percent change from | |
| IPE 4 g/day (n = 76) | | | IPE 2 g/day (n = 73) | | | Placebo (n = 75) | | | baseline | |
| | | Change | | | Change | | | Change | IPE | IPE |
| | End-of- | from | | End-of- | from | | End-of- | from | 4 g/day vs | 2 g/day vs |
| Baseline | treatment | baseline | Baseline | treatment | baseline | Baseline | treatment | baseline | placebo, | placebo, |
| (SD) | (SD) | (SE) | (SD) | (SD) | (SE) | (SD) | (SD) | (SE) | %, P | %, P |

Plasma

| | | | | | | | | | | | |
|---|---|---|---|---|---|---|---|---|---|---|---|
| Saturated | 35.9 (3.9) | 34.8 (3.5) | −1.2 (0.5) | 36.2 (3.8) | 35.8 (3.7) | −0.3 (0.5) | 35.6 (3.0) | 35.7 (3.2) | −0.2 (0.5) | −2.8 | −0.4 |
| n = 49, 56, 49 | | | | | | | | | | 0.1049 | 0.8076 |
| Monounsaturated | 30.1 (5.3) | 28.7 (4.0) | −1.7 (0.5) | 30.7 (5.1) | 29.5 (4.9) | −1.4 (0.5) | 31.4 (4.7) | 32.3 (5.2) | 0.9 (0.5) | −7.7 | −7.4 |
| n = 49, 56, 49 | | | | | | | | | | 0.0008 | 0.0009 |
| Total omega-6 | 30.8 (7.2) | 29.8 (6.2) | −0.9 (0.7) | 30.0 (7.1) | 29.8 (6.8) | −0.2 (0.7) | 30.0 (5.8) | 29.1 (5.4) | −0.9 (0.7) | 0.5 | 3.2 |
| n = 49, 56, 49 | | | | | | | | | | 0.8846 | 0.3044 |
| Total omega-3 | 3.2 (1.2) | 6.7 (1.9) | 3.8 (0.2) | 3.0 (1.0) | 4.8 (1.3) | 2.0 (0.2) | 2.9 (0.9) | 3.0 (1.1) | 0.2 (0.2) | 132.7 | 68.4 |
| n = 49, 56, 49 | | | | | | | | | | <0.0001 | <0.0001 |

RBCs

| | | | | | | | | | | | |
|---|---|---|---|---|---|---|---|---|---|---|---|
| Saturated | 43.5 (1.4) | 44.5 (2.5) | 0.6 (0.4) | 43.8 (1.8) | 44.4 (3.0) | 0.3 (0.4) | 44.2 (4.2) | 43.8 (2.6) | −0.3 (0.4) | 1.9 | 1.1 |
| n = 50, 56, 49 | | | | | | | | | | 0.1145 | 0.3278 |
| Monounsaturated | 19.6 (2.2) | 19.5 (1.9) | −0.2 (0.3) | 20.2 (2.8) | 20.0 (1.9) | −0.1 (0.3) | 20.4 (3.0) | 20.8 (2.9) | 0.6 (0.3) | −3.9 | −3.1 |
| n = 50, 56, 49 | | | | | | | | | | 0.0154 | 0.0435 |
| Total omega-6 | 30.4 (2.2) | 25.5 (2.9) | −4.7 (0.5) | 29.8 (3.2) | 27.0 (3.3) | −2.9 (0.4) | 29.1 (5.1) | 29.5 (3.6) | −0.1 (0.5) | −17.3 | −13.2 |
| n = 50, 56, 49 | | | | | | | | | | <0.0001 | 0.0014 |
| Total omega-3 | 6.6 (1.7) | 10.4 (2.3) | 4.1 (0.3) | 6.2 (1.6) | 8.7 (2.2) | 2.6 (0.3) | 6.2 (2.2) | 5.9 (2.0) | −0.2 (0.3) | 53.4 | 23.8 |
| n = 50, 56, 49 | | | | | | | | | | 0.0008 | 0.1183 |

Table 3 Notes:
Mean and standard deviation are reported for baseline and end-of-treatment values; least-squares mean and standard error are reported for changes from baseline and placebo-adjusted changes from baseline.
^{a}Total = sum of omega-3, omega-6, monounsaturated, and saturated fatty acids.
Saturated fatty acids = sum of myristic, palmitic, stearic, arachidic, behenic, and lignoceric acids.
Monounsaturated fatty acids = myristoleic, palmitoleic, cis-vaccenic, oleic, gondoic/gadoleic, erucic, and nervonic acids.
Omega-6 fatty acids = sum of linoleic, γ-linolenic, eicosadienoic, dihomo-γ-linolenic, omega-6 arachidonic, adrenic, and omega-6 docosapentaenoic acids.
Omega-3 fatty acids = sum of α-linolenic, stearidonic, eicosatrienoic, omega-3 arachidonic, eicosapentaenoic, omega-3 docosapentaenoic, and docosahexaenoic acids.

What is claimed is:

1. A method of increasing a concentration of EPA in plasma or RBCs of a subject having baseline fasting triglycerides of at least about 500 mg/dl, the method comprising administering to the subject a pharmaceutical composition comprising about 1 g to about 4 g of EPA per day, wherein upon administering the composition to the subject daily for a period of 12 weeks, the subject exhibits an increase in the concentration of EPA in plasma and/or RBCs of at least 100% compared to a control subject who is not administered EPA, wherein the control subject also has baseline fasting triglycerides of at least about 500 mg/dl.

2. The method of claim 1 wherein the increase is at least 200%, at least about 400%, or at least about 600%.

3. The method of claim 1, wherein the subject exhibits no increase, no significant increase in, or a decrease in a concentration of DHA in plasma and/or RBCs.

4. The method of claim 1, wherein the subject is administered about 2 g/day or about 4 g/day of EPA.

5. The method of claim 1, wherein the subject further exhibits no increase, no substantial increase, or a reduction in plasma and/or RBC levels of DHA compared to the control subject.

6. A method of reducing a ratio of arachidonic acid to EPA (AA/EPA) in plasma or RBCs of a subject having baseline fasting triglycerides of at least about 500 mg/dl, the method comprising administering to the subject a pharmaceutical composition comprising about 1 g to about 4 g of EPA per day, wherein upon administering the composition to the subject daily for a period of 12 weeks, the subject exhibits a decrease in AA/EPA in plasma and/or RBCs of at least 50% compared to a control subject who is not administered EPA, wherein the control subject also has baseline fasting triglycerides of at least about 500 mg/dl.

7. The method of claim 6, wherein the decrease in AA/EPA in plasma and/or RBCs is at least 70% or at least 75%.

8. The method of claim 6, wherein the decrease includes a decrease in AA/EPA in plasma of at least 75% or at least 90%.

9. The method of claim 6, wherein the decrease includes a decrease in AA/EPA in RBCs of at least 80%, at least 90%, or at least 95%

10. The method of claim 6, wherein the subject is administered about 2 g/day or about 4 g/day of EPA.

11. The method of claim 6, wherein the subject further exhibits no increase, no substantial increase, or a reduction in plasma and/or RBC levels of DHA compared to the control subject.

12. A method of reducing a ratio of omega-3 fatty acids to omega-6 fatty acids (n-3/n-6) in plasma or RBCs of a subject having baseline fasting triglycerides of about 200 mg/dl to about 500 mg/dl and on stable statin therapy, the method comprising administering to the subject a pharmaceutical composition comprising about 1 g to about 4 g of EPA per day, wherein upon administering the composition to the subject daily for a period of 12 weeks, the subject exhibits a decrease in n-3/n-6 in plasma and/or RBCs of at least 50% compared to a control subject who is not administered EPA, wherein the control subject also has baseline fasting triglycerides of at least about 500 mg/dl.

13. The method of claim 12, wherein the decrease in n-3/n-6 in plasma and/or RBCs is at least 60% or at least 100%.

14. The method of claim 12, wherein the decrease includes a decrease in n-3/n-6 in plasma of at least 60%, at least 100%, or at least 140%.

15. The method of claim 12, wherein the decrease includes a decrease in n-3/n-6 in RBCs of at least 60%, at least 80%, or at least 100%.

16. The method of claim 12, wherein the omega-3 fatty acids include one or more of: α-linolenic acid, stearidonic acid, eicosatrienoic acid, ω-3 arachidonic acid, eicosapentaenoic acid, ω-3 docosapentaenoic acid and docosahecaenoic acid.

17. The method of claim 12, wherein the omega-6 fatty acids include one or more of: linoleic acid, γ-linolenic acid, eicosadienoic acid, dihomo-γ-linolenic acid, ω-6 arachidonic acid, adrenic acid, and ω-6 docosapentaenoic acid.

18. The method of claim 12, wherein the subject is administered about 2 g/day or about 4 g/day of EPA.

19. The method of claim 12, wherein the subject further exhibits no increase, no substantial increase, or a reduction in plasma and/or RBC levels of DHA compared to the control subject.

20. A method of increasing a ratio of omega-3 fatty acids to total fatty acids in plasma or RBCs of a subject having baseline fasting triglycerides of at least about 500 mg/dl, the method comprising administering to the subject a pharmaceutical composition comprising about 1 g to about 4 g of EPA per day, wherein upon administering the composition to the subject daily for a period of 12 weeks, the subject exhibits an increase in the ratio of omega-3 fatty acids to total fatty acids in plasma and/or RBCs of at least 20% compared to a control subject who is not administered EPA, wherein the control subject also has baseline fasting triglycerides of at least about 500 mg/dl.

21. The method of claim 20, wherein the increase is at least about 50%, at least about 60%, or at least about 130%.

22. The method of claim 20, wherein the increase includes an increase in the ratio in plasma of at least 60% or at least about 130%.

23. The method of claim 20, wherein the increase includes an increase in the ratio in RBCs of at least 20% or at least about 50%.

24. The method of claim 20, wherein the omega-3 fatty acids include one or more of: α-linolenic acid, stearidonic acid, eicosatrienoic acid, ω-3 arachidonic acid, eicosapentaenoic acid, ω-3 docosapentaenoic acid and docosahecaenoic acid.

25. The method of claim 20, wherein the total fatty acids include one or more of:
α-linolenic acid, stearidonic acid, eicosatrienoic acid, ω-3 arachidonic acid, eicosapentaenoic acid, ω-3 docosapentaenoic acid, docosahecaenoic acid, linoleic acid, γ-linolenic acid, eicosadienoic acid, dihomo-γ-linolenic acid, ω-6 arachidonic acid, adrenic acid, ω-6 docosapentaenoic acid, myristoleic acid, palmitoleic acid, cis-vaccenic acid, oleic acid, gondoic/gadoleic acid, erucic acid, nervonic acid, myristic acid, palmitic acid, stearic acid, arachidic acid, behenic acid and lignoceric acid.

26. The method of claim 20, wherein the subject is administered about 2 g/day or about 4 g/day of EPA.

27. The method of claim 20, wherein the subject further exhibits no increase, no substantial increase, or a reduction in plasma and/or RBC levels of DHA compared to the control subject.

28. A method of decreasing a ratio of omega-6 fatty acids to total fatty acids in plasma or RBCs of a subject having baseline fasting triglycerides of at least about 500 mg/dl, the method comprising administering to the subject a pharmaceutical composition comprising about 1 g to about 4 g of EPA per day, wherein upon administering the composition to the subject daily for a period of 12 weeks, the subject exhibits a decrease in the ratio of omega-6 fatty acids to total fatty acids in plasma and/or RBCs of at least 5% compared to a control subject on stable statin therapy without EPA, wherein the control subject also has baseline fasting triglycerides of about 200 mg/dl to about 500 mg/dl.

29. The method of claim 28, wherein the decrease is at least about 10%, at least about 13%, or at least about 17%.

30. The method of claim 28, wherein the decrease includes a decrease in the ratio in RBCs of at least 13% or at least about 17%.

31. The method of claim 28, wherein the omega-6 fatty acids include one or more of: linoleic acid, γ-linolenic acid, eicosadienoic acid, dihomo-γ-linolenic acid, ω-6 arachidonic acid, adrenic acid, and ω-6 docosapentaenoic acid.

32. The method of claim 28, wherein the total fatty acids include one or more of:
α-linolenic acid, stearidonic acid, eicosatrienoic acid, ω-3 arachidonic acid, eicosapentaenoic acid, ω-3 docosapentaenoic acid, docosahecaenoic acid, linoleic acid, γ-linolenic acid, eicosadienoic acid, dihomo-γ-linolenic acid, ω-6 arachidonic acid, adrenic acid, ω-6 docosapentaenoic acid, myristoleic acid, palmitoleic acid, cis-vaccenic acid, oleic acid, gondoic/gadoleic acid, erucic acid, nervonic acid, myristic acid, palmitic acid, stearic acid, arachidic acid, behenic acid and lignoceric acid.

33. The method of claim 28, wherein the subject is administered about 2 g/day or about 4 g/day of EPA.

34. The method of claim 28, wherein the subject further exhibits no increase, no substantial increase, or a reduction in plasma and/or RBC levels of DHA compared to the control subject.
